(19) United States
(12) Patent Application Publication
(10) Pub. No.: US 2006/0251612 A1
(43) Pub. Date: Nov. 9, 2006

(54) Bioresorbable cyanoacrylate adhesives

(76) Inventors: Dimiter Kotzev, Corby (GB); Vega Kotzev, Corby (GB)

Correspondence Address: LEACH PATENT SERVICES, 5 EAST ALDINE DRIVE, HOCKESSIN, DE, 19707, US

(21) Appl. No.: 11/124,831

(22) Filed: May 9, 2005

(51) Int. Cl. A61K 38/39; A61K 31/785; A61K 31/716

(52) U.S. Cl. 424/78.27; 514/54; 514/2

(57) ABSTRACT

Bioresorbable cyanoacrylate-based adhesives containing body fluid soluble additives are disclosed. The body fluid soluble additives are compounds which are insoluble in cyanoacrylate monomer but which are readily dissolved out of the cured adhesive in application. The resulting pores and channels which provide ready pathways for connective tissue ingrowth and facilitating quick wound healing. The adhesives of the invention are useful for wound and incision closure, implants, medical device fixation, embolic agents and other general medical applications.

### BACKGROUND OF THE INVENTION

1. Field of the Invention

This invention relates to cyanoacrylate adhesives, and more particularly, to bioresorbable cyanoacrylate tissue adhesive compositions and to methods for making and using these compositions. The compositions are useful in medical applications, including, but not limited to, wound and surgical incision closure, implants, medical device fixation, sealants and void fillers, embolic agents and other general medical applications.

2. Background

Surgical incisions and wounds can be closed by three general methods—suturing, stapling and adhesive bonding.

U.S. Pat. No. 5,578,046 teaches that sutures are bioabsorbable when the material that they are made from is capable of being broken down into smaller constituents, which can be metabolized and excreted by the living organism. Such materials are useful for temporarily holding tissues in a desired position during healing and are absorbed by the organism after a period of time. The teachings of U.S. Pat. No. 5,578,046 as well as the patents and literature in turn referenced by U.S. Pat. No. 5,578,046 are incorporated as reference herein.

Wound suturing has the advantage of producing bioabsorbable, non-toxic degradation products. It however also has disadvantages. Suturing requires time and skill. It causes additional trauma to the tissue by piercing and does not provide a hermetic closure.

Cyanoacrylates possess the unique property to bond living tissue. They have been widely and successfully tested for closing wounds and incisions, especially in cases where suturing does not provide satisfactory results. See Lijoi A. et al, “Subacute left ventricular free wall rupture complicating acute myocardial infarction. Successful surgical repair with a sutureless technique”, J. Cardiovascular Surgery, December 1996, 37(6), 627-630; Tebala G. D. et al, “The use of cyanoacrylate tissue adhesive in high-risk intestinal anastomoses”, Surgery Today, 1995, 25(12), 1069-72 and Zaki I. et al, “Split skin grafting on severely damaged skin. A technique using absorbable tissue adhesive”, J. of Dermatologic Surgery and Oncology, December 1994, 20(12), 827-9.

Cyanoacrylate tissue adhesive have the following advantages over suturing: they save time; they can bond difficult to suture tissues; they can provide a hermetic closure; they have hemostatic action; they produce better cosmetic results; they are indispensable in emergencies.

A major disadvantage of cyanoacrylate adhesives is that one of the degradation products is formaldehyde, which is toxic to the surrounding tissues (see Pani K. C. et al, “The degradation of n-butyl alpha-cyanoacrylate tissue adhesive. II.”, Surgery, March 1968, 63(3), 481-9). For this reason cyanoacrylates have not found favor with the FDA for internal tissue closure. Only topical skin closure applications have been FDA approved.

U.S. Pat. Nos. 6,224,622 and 6,103,778, published U.S. patent application 2003/0069535 and WO2004084963 disclose bioabsorbable cyanoacrylate adhesives which contain bioabsorbable polymer additives and their teachings are incorporated into the present application by reference. WO2004084963 further describes and claims a porous adhesive with pores between 10 and 500 nm. These pores however are too small to allow connective tissue ingrowth and healing. These cited references disclose homogeneous solutions of bioabsorbable polymers in cyanoacrylate monomers, which, following polymerization, cannot yield large enough pores required for connective tissue growth. Although the references provide an improved rate of bioabsorption, a much higher rate is needed in order that cyanoacrylate adhesives become competitive with sutures in closing wounds and incisions.

US published application 20020086047 teaches that nutrient media and oxygen can pass through membranes with pore size of 0.5 to 3 microns. This pore size is insufficient however for growth of connective tissue cells. 1 to 5 mm channels are, for example, required for growth of naturally occurring nerves.

A. Coombes (“Polymeric Matrices for Guiding Cell Behavior and Organization in Tissue Engineering”, Medical Polymers 2003, Dublin, Ireland, paper 19, p.167-1 71) teaches that the microstructure and architecture of an implanted scaffold exert profound effect on cell behavior and tissue organization by providing pathways, for example, for guided tissue regeneration within and over the material. The size of micropores and the structure of the interconnections determine the extent of tissue ingrowth whilst micropores allow exchange of nutrients and metabolites and may also provide a favorable surface topography for cell attachment. Precise control is required over pore size and geometry since these factors are known to be key determinants of the type of tissue ingrowth. For example, bony ingrowth was found to predominate in porous polymethylmethacrylate implanted in bone when the pore size was around 450 microns. Connective tissue formed when the pore size was around 100 microns and extensive vascular infiltration was only observed with pores around 1000 microns. Structures comprising macropores (150 -300 microns) highly interconnected by micropores (less than 50 microns) have been found to be conducive to ingrowth of fibrinocartilaginous tissue in polyurethane implants.

U.S. Pat. No. 4,594,407 describes a prosthetic device having pores with interspatial dimensions of about 200 microns which allows several layers of cells to form through and within each pore. The invading fibroblast cells commence formation of collagen leading to connective tissue while macrophages and extracellular enzymes degrade the material, and newly formed capillary vessels penetrate the prosthesis and provide blood containing oxygen and nutrients which further the formation of organized tissue, around as well as within the prosthetic device.

### BRIEF SUMMARY OF THE INVENTION

One embodiment of the invention is directed to a method for making a bioresorbable tissue adhesive composition comprising the step of dispersing into a cyanoacrylate monomer or blend of cyanoacrylate monomers a body fluid soluble additive.

Another embodiment is directed to bioresorbable tissue adhesives made by this method.

Another embodiment of the invention is directed to a method for making a bioresorbable cyanoacrylate tissue adhesive composition comprising the additional step of dissolving one or more copolymers, the copolymers derived from glycolide and one or more monomers as described in U.S. Pat. No. 6,224,622.

Another embodiment is directed to a bioresorbable tissue adhesives made by this method.

Other embodiments and advantages of the invention are set forth in part in the description which follows, and in part, will be obvious from this description, or may be learned from the practice of the invention.

### DETAILED DESCRIPTION OF THE INVENTION

As embodied and broadly described herein, the present invention is directed to cyanoacrylate-based tissue adhesives which combine the advantages of bioabsorbable suturing with the advantages of adhesive bonding. The compositions of the present invention are quickly bioresorbed and provide hermetic closure and hemostatic action.

This invention relates to cyanoacrylate adhesives, and more particularly, to bioresorbable cyanoacrylate tissue adhesive compositions and to methods for making and using these compositions. The compositions are useful in medical applications, including, but not limited to, wound and surgical incision closure, implants, medical device fixation, sealants and void fillers, embolic agents and other general medical applications. The compositions of the invention comprise cyanoacrylate monomers and at least one body fluid soluble additive.

A degradable material is a material that can decompose, degenerate, degrade, depolymerize, or otherwise reduce the molecular weight of the starting compound(s) such that the resulting compound(s) is (are) soluble in water or, if insoluble, can be suspended in a body fluid and transported away from the implantation site without clogging the flow of the body fluid.

A resorbable material is a material that is soluble, degradable as defined above, or is an aggregate of soluble and/or degradable material(s) with insoluble material(s) such that, with the resorption of the soluble and/or degradable materials, the residual insoluble materials are of sufficiently fine size that they can be suspended in a body fluid and transported away from the site without clogging the flow of the body fluid. Ultimately the particles are eliminated from the body either by excretion in perspiration, urine or feces, or dissolved, degraded, corroded, or otherwise metabolized into soluble components that are excreted from the body.

A bioresorbable material is a resorbable material that is biocompatible.

A biocompatible material is a material that is compatible with living tissues or a living system, non-toxic or non-injurious and does not cause immunological reaction or rejection.

The term bioresorbable is used herein to mean not only biodegradable but that the degradation products, formed in vivo from those materials, are metabolizable by the mammalian body, without any toxic or otherwise harmful side effects.

The present invention overcomes the problems and disadvantages associated with current cyanoacrylate adhesives and provides compositions useful as bioresorbable tissue adhesives. This is achieved by incorporating into the adhesive one or more body fluid soluble additives with predetermined form and size, which is not substantially soluble into the cyanoacrylate. Upon polymerization of the adhesive the soluble additive comes in contact with the tissue fluids and is quickly dissolved and removed from the adhesive layer. The formed voids form interconnected pore structures, which provides pathways for connective tissue ingrowth, connecting the joined tissue surfaces and facilitating quick wound healing. In another aspect the new surface area created following the removal of the soluble component facilitates the biodegradation of the remaining cyanoacrylate polymer, making it more accessible to the body fluids and metabolites.

As used herein a body fluid soluble material is a material that has water solubility such that upon exposure to a body fluid an amount of the material will dissolve or erode over time. “Body fluid” herein refers to fluids in the body of a mammal including, but not limited to, blood, urine, saliva, lymph, plasma, gastric, biliary, or intestinal fluids, seminal fluids, and mucosal fluids or humors.

The terms body fluid soluble, water-soluble, and soluble are used herein interchangeably and have the meaning defined for body fluid soluble.

The size of the water-soluble additive particles can be chosen depending on the tissues that are to be bonded, so as to be maximally suitable for the area of application, i.e. whether joining soft tissue, bone tissue, parenchymal tissue, nerve tissue, skin etc.

Generally the water-soluble additive embedded in the bioresorbable cyanoacrylate material facilitates the resorption of the bulk material at a controllable resorption rate upon contact with a body fluid. The bioabsorbable bulk material resorbs at a different and faster rate than when it would if there were no particles embedded in the bulk material. The resorption rate of the biabsorbable material can be controlled by varying the chemical and physical properties of the particles, their size, shape, amount and distribution, etc. The resorbable particles generally resorb at a different and faster rate than the bioresorbable bulk material. Depending on the body fluid soluble additive chosen, its particle size, and the amount used, the rate of resorption is typically characterized by a loss of at least about 6% of the mass of the adhesive within the first 7 days after application. Preferably, the rate of resorption is typically characterized by a loss of at least about 10% of the mass of the adhesive within the first 7 days after application. More preferably, the rate of resorption is typically characterized by a loss of at least about 20% of the mass of the adhesive within the first 7 days after application.

The resorbable particles may include a swelling agent, a hydrolysable agent, or a soluble agent or a combination thereof. These agents may be organic compounds, polymeric compounds, soluble or degradable inorganic compounds, and/or organic or inorganic crystals or powder aggregates. The most preferred additives are organic or inorganic crystals or powder aggregates, which are not soluble in the cyanoacrylate monomer(s).

The size of the particles may be from about 0.5 microns to about 1 mm. The preferred size is from 10 microns to 500 microns. The most preferred size is from 50 microns to 300 microns. The distribution of the particles need not be uniform. The volume percentage of the particles in the bulk material can be between 1 and about 50%. A preferred volume percentage of the particles in the bulk material can be between about 5% and about 40%. A more preferred volume percentage of the particles in the bulk material can be between about 10% and about 40%.

Generally, the sizes of the pores and pathways created by resorption of the particles will be similar to the sizes of the particles used in the adhesive. The size of the pores may be from about 0.5 microns to about 1 mm. The preferred size of the pores and pathways is from about 10 microns to about 500 microns. The more preferred size of the pores and pathways is from about 50 microns to about 300 microns. The distribution of the pores and pathways need not be uniform.

Examples of substances which may be used as body fluid soluble additives include 2-hydroxycarpoic acid, 3-hydroxybutyric acid, 4-O-(β-galactosyl)-D-glucitol, agar, albumin, alginic acid, alpha-D-glucose, aspertic acid derivatives, barium sulfate, calcium citrate, calcium lactate, calcium phosphate, calcium propionate, carboxymethyl cellulose, carboxymethyl cellulose sodium salt, carboxymethyl chitosan, carboxymethyl starch, cationic starch, cellulose, cellulose acetate, chitin, chitosan, chondroitin-4-sulfate, chondroitin-6-sulfate, citric acid and collagen. Further examples include copolymers of N-(2-hydroxypropyl)methacrylamide, Debrisan® beads from Pharmacia, Dermatan sulfate, dextran, dextran based biodegradable beads from American Biosciences under trade names of Sephadex®t, Sepharose®, Sephacel®, DL-aspartic acid, ferrous gluconate, fibrin, gelatin, glucono-delta-lactone, glutamic acid derivatives, guar, heparan sulfate, heparin, heparin sulfate, hyaluronic acid, inulin, keratan, lactic acid, lithium hydroxide, magnesium hydroxide, magnesium lactate, magnesium oxide, pectinic acid, poly(1,4-butylene succinate) extended with 1,6-iisocyanatohexane, poly(2,3-butylene fumarate), poly(2,3-butylene hydroxysuccinate) and poly(2,3-butylene succinate). Still further examples include poly(amino acids), poly(malic)acid, poly[di(carboxylatophenoxy)phosphazene], polyacetals, polyacrylic acid, polyacrylic acid copolymers, polyalkylene oxalates, polyalkylene succinates, polyanhydrides, polyaspartate, polycarbonates, polydioxanones, polyesteramides, polyesters, polyethylene amine, polyethylene glycol, polyhydroxybutyrates, polyhydroxybutyric acid, polyhydroxycellulose, polyhydroxyvalerates, polyhydroxyvaleric acid, polyketals, polymethacrylic acid, polyorthoethers, polyoxyethylenesorbitan monolaurate, polyphosphazenes, polypropylene glycol, polysaccharides, polyurethanes, potassium acetate, potassium gluconate, sodium acetate, sodium alginate, starch, triethyl citrate, xanthan gum, ε-caprolactone, ε-hydroxycaproic acid and ω-hydroxybutyric acid.

Typical body fluid soluble additives include calcium L(+) lactate, magnesium L(+) lactate, gluconic acid delta lactone, c-caprolactone, soluble starch, gelatin, innulin from chicory leaf, 2-hydroxycaproic acid and mixtures thereof. Preferred body fluid soluble additives include mixtures of the calcium and magnesium salts of L(+) lactic acid, gelatin, innulin, and mixtures of the above with ε-caprolactone and with 2-hydroxycaproic acid. Most preferred are mixtures of the calcium and magnesium salts of L(+) lactic acid with F-caprolactone and mixtures of innulin with F-caprolactone.

It is essential that the soluble additives of the present invention are themselves essentially insoluble in the cyanoacrylate monomer(s). Some of the suitable compounds are inherently insoluble. Body fluid soluble compounds which are soluble in cyanoacrylate can be rendered insoluble in cyanoacrylate. Similarly suitable additives which otherwise are incompatible or have a destabilizing effect on cyanoacrylates can be rendered compatible or the destabilizing effect removed by coating the particles with material which prevents their direct contact with cyanoacrylates.

In one embodiment, the bioresorption is controlled by the rate of dissolving of the particles upon contact with the body fluid removing them from the adhesive layer. Dissolution of the particles creates voids in the matrix of the adhesive and an increased porosity. As a consequence, the diffusion rate of the fluid into the bulk material increases, thereby promoting resorption and the eventual degradation of the bulk cyanoacrylate material.

In another embodiment, the bioresorption is controlled by the hydrolysis of the particles upon contact with a body fluid producing soluble by-products. Hydrolysis of the particles into soluble by-products results in voids in the matrix of the adhesive layer and an increased porosity. As a consequence, the diffusion rate of the fluid into the bulk material increases, thereby promoting resorption and the eventual degradation of the bulk cyanoacrylate material.

In another embodiment the bioresorption is controlled by the swelling of the embedded particles upon contact with the body fluid, which leads to weakening of the matrix structure of the bulk cyanoacrylate material and its eventual break up into small fragments. In addition fragmentation into small pieces also results in an increased contact area of the bulk material with the body fluid. The consequence is increased fluid diffusion rate that promotes resorption.

In another embodiment the body fluid soluble additive is substantially removed from the adhesive layer in the first week after the application of the adhesive providing the pathways for tissue growth and healing.

In another embodiment the body fluid soluble additive is substantially removed from the adhesive layer in the first few days after the application of the adhesive providing the pathways for tissue growth and healing.

In another embodiment the body fluid soluble additive is substantially removed from the adhesive layer in the first few hours after the application of the adhesive providing the pathways for tissue growth and healing.

The cyanoacrylate monomer or monomers can be selected from the group consisting of alkyl 2-cyanoacrylate, alkenyl 2-cyanoacrylate, alkoxyalkyl 2-cyanoacrylate, or carbalkoxyalkyl 2-cyanoacrylate. The alkyl group of the cyanoacrylate monomer or monomers preferably has 1 to 16 carbon atoms, and includes cycloalkyl functionality. Suitable cyanoacrylates include for example methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate and 2-propoxyethyl 2-cyanoacrylate.

In some embodiments, the compositions of the invention may further comprise a copolymer. Suitable copolymers are described in U.S. Pat. No. 6,224,622, the disclosure of which has already been incorporated by reference. Typical copolymers include copolymers of one or more cyanoacrylate monomers with glycolide, lactide, ε-caprolactone, dioxanone and trimethylene carbonate. Other suitable copolymers being copolymers of glycolide with lactide, F-caprolactone, dioxanone and trimethylene carbonate. The cyanoacrylate monomer or monomers can be selected from the group consisting of alkyl 2-cyanoacrylate, alkenyl 2-cyanoacrylate, alkoxyalkyl 2-cyanoacrylate, or carbalkoxyalkyl 2-cyanoacrylate. The alkyl group of the cyanoacrylate monomer or monomers preferably has 1 to 16 carbon atoms, and includes cycloalkyl functionality. Suitable cyanoacrylates include for example methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate and 2-propoxyethyl 2-cyanoacrylate.

The present invention is useful in medical applications, including veterinary and other applications where a bioresorbable bond is desired. Compositions of the invention may be used to bond tissue to tissue, tissue to a foreign object such as an implant, or even two foreign objects to each other. They can also be used as implants.

The bioresorbable cyanoacrylate adhesives of the present invention are obtained by dispersing one or more of the above-described body fluid soluble additives into one or more of the above-described cyanoacrylate monomers. Unexpectedly high amounts of additives can easily be dispersed into the composition by mixing at room temperature.

The bioresorbable cyanoacrylate adhesives of the present invention can be stabilized against premature polymerization with anionic and free-radical polymerization inhibitors. Anionic polymerization inhibitors, known in the art include soluble acidic gases (for example sulfur dioxide), and phosphoric, carboxylic and organic sulphonic acids. Free-radical polymerization inhibitors include hydroquinone, t-butyl catechol, hydroxyanisole, butylated hydroxyanisole and butylated hydroxytoluene.

The present invention provides a method of treating living tissue, comprising selecting a cyanoacrylate monomer for treatment of the tissue, selecting a body fluid soluble additive and amount for a desired resorption rate and pore size, and applying to living tissue the adhesive composition to form a resorbable adhesive polymer.

The bioabsorbable cyanoacrylate adhesives of the present invention may contain any additional additives necessary to impart desired properties to the adhesive including, but not limited to, viscosity, color, X-ray opacity, as well as antimicrobial agents, antibiotics, growth promoting factors, anti-cancer drugs, immune system enhancing drugs.

For example dyes contemplated for use in the present invention are D&C Violet No. 2, D&C Green No. 6, carbon black and bone black.

For example growth factors contemplated for use in the adhesives of the present invention are fibroblast growth factor, bone growth factor, epidermal growth factor, platelet derived growth factor, macrophage derived growth factor, alveolar derived growth factor, monocyte derived growth factor, magainin, and so forth.

The adhesive compositions of the present invention can be heat sterilized as disclosed in U.K. Pat. GB 2306469B, U.S. Pat. No. 5,874,044 and U.S. Pat. No. 6,136,326, the disclosures of which are herein incorporated by reference.

Applications of the present invention include, but are not limited to, wound closure (including surgical incisions and other wounds), adhesives for medical devices, implants, sealants and void fillers in human and animal medical applications and embolic agents.

The following examples are offered to illustrate embodiments of the invention, and should not be viewed as limiting the scope of the invention.

Materials

The Materials Used are Summarized in Table 1

TABLE 1
Materials

| Trade name | Chemical name | Manufacturer | Composition |
|---|---|---|---|
| NBCA | n-butyl 2- | Chemence Medical, | 99.9% |
| | cyanoacrylate | Alpharetta, Georgia | |
| Puracal PP/USP | Calcium salt | Purac America Inc., | Particle size |
| | of natural L(+) | Lincolnshire, IL | 75μ-424μ |
| | lactic acid | | |
| Puramex MG | Magnesium salt | Purac America Inc., | |
| | of natural L(+) | Lincolnshire, IL | |
| | lactic acid | | |
| Gluconal GDL | Gluconic acid | Purac America Inc., | |
| | delta lactone | Lincolnshire, IL | |
| | ε-caprolactone | Sigma-Aldrich, | |
| | | Saint Louis, MO | |
| Purac Powder 60 | 60% lactic acid | Purac America Inc., | Particle size |
| | 40% calcium | Lincolnshire, IL | less than 710μ |
| | lactate | | |
| | Soluble starch | Sigma-Aldrich, | |
| | | Saint Louis, MO | |
| | Gelatin | Sigma-Aldrich, | 300 bloom |
| | | Saint Louis, MO | |
| | Innulin from | Sigma-Aldrich, | |
| | chicory leaf | Saint Louis, MO | |
| | 2-Hydroxy | Sigma-Aldrich, | |
| | caproic acid | Saint Louis, MO | |

### EXAMPLE 1

### Preparation of Adhesives

Bioabsorbable cyanoacrylate tissue adhesive compositions were obtained by mixing by stirring a measured amount of additive into n-butyl 2-cyanoacrylate (NBCA) at room temperature until homogeneous dispersion was obtained. Freshly stirred adhesives were applied for weight loss and adhesive strength measurements. The quantities of additive(s) and cyanoacrylate are shown in Table 2.

TABLE 2
Adhesive formulations

| Adhesive | Quantity of | | Quantity of |
|---|---|---|---|
| No. | NBCA (g) | Additive | additive (g) |
| 1 | 6.7 | Puracal PP/USP | 3.3 |
| 2 | 6.7 | Puramex MG | 3.3 |
| 3 | 6.7 | Gluconal GDL | 3.3 |
| 4 | 6.7 | Purac Powder 60 | 1.65 |
| | | ε-caprolactone | 1.65 |
| 5 | 6.7 | Soluble starch | 3.3 |
| 6 | 10 | none | — |
| 7 | 6.7 | Gelatin | 3.3 |
| 8 | 6.7 | Innulin | 1.65 |
| | | 2-Hydroxy caproic acid | 1.65 |
| 9 | 6.7 | Innulin | 1.65 |
| | | ε-caprolactone | 1.65 |

### EXAMPLE 2

### In-vitro Mass Loss of Adhesive Film

Cured adhesive film was prepared by spreading two drops (0.07 g) of adhesive on the surface of a polished circular KBr plate of 2.5 cm diameter. On top of it identical KBr plate was positioned and the adhesive left to cure. 24 hours later the adhesively joined plates were placed in water at room temperature until the KBr plates were dissolved. The adhesive film was dried in air, followed by drying in a vacuum oven for 4 hours at 37° C., followed by conditioning for 12 hours in a desiccator cabinet. The weight of the adhesive film was measured on an analytical balance. The adhesive film was placed in Phosphate buffer solution of pH=7.2 kept at 37 (±0.5)° C. Samples were removed from the buffer solution at measured periods of time, washed with deionized water, dried at 37° C. under vacuum for 4 hours and placed in a desiccator cabinet for 12 hours. Then the weight of the sample was measured and the weight loss calculated. The results are presented in Table 3. For comparison an adhesive film of unmodified NBCA was tested alongside (adhesive No. 6)

TABLE 3
In-vitro weight loss of adhesive films

| Days | | | | | | | | | |
|---|---|---|---|---|---|---|---|---|---|
| in | | | | | | | | | |
| buffer | Weight loss (%) | | | | | | | | |
| at | Adh | Adh | Adh | Adh | Adh | Adh | Adh | Adh | Adh |
| 37° C. | 1 | 2 | 3 | 4 | 5 | 6 | 7 | 8 | 9 |
| 1 | 1.8 | 6.2 | 5.8 | 26.2 | 1.7 | 1.0 | 10.0 | 12.9 | 10.2 |
| 2 | 2.6 | 8.7 | 7.6 | 28.1 | 3.1 | 2.2 | | 15.1 | 14.7 |
| 3 | 5.6 | 11.1 | 9.1 | 28.7 | 3.8 | 3.5 | 23.1 | 18.0 | 19.9 |
| 5 | 6.0 | 13.4 | 9.4 | 29.2 | 4.0 | 2.7 | 26.9 | | 23.9 |
| 6 | 6.6 | 14.4 | 9.6 | 29.2 | | 3.0 | | 18.8 | 25.3 |
| 13 | 7.4 | 18.0 | 10.0 | 29.8 | 4.5 | 3.3 | 27.6 | 20.8 | 28.8 |
| 20 | 7.9 | 18.7 | 10.8 | 30.3 | 5.0 | 3.8 | 28.0 | 21.3 | 29.4 |
| 27 | 8.2 | 19.2 | 11.3 | 31.0 | 5.7 | 4.1 | 28.5 | 21.8 | 30.0 |
| 34 | 8.8 | 19.7 | 12.0 | 31.6 | 6.2 | 4.7 | 28.9 | 22.3 | 30.5 |
| 42 | 9.2 | 20.2 | 12.8 | 32.2 | 7.0 | 5.2 | 29.4 | 22.9 | 31.0 |
| 72 | 11.0 | 21.0 | 14.0 | 34.8 | 7.5 | 6.2 | 30.7 | 25.0 | 32.5 |
| 114 | 14.0 | 22.6 | 17.0 | 38.3 | 9.4 | 7.2 | 31.6 | 27.5 | 35.5 |
| 171 | 16.5 | 25.8 | 19.0 | 42.5 | 12.0 | 8.0 | 33.4 | 31.6 | 39.5 |

It can clearly be seen that the adhesives containing soluble additives lose substantial amount of mass during the first week and especially during the first days after immersion in buffer solution. In some cases (adhesives 4 and 9) as much as 29% and 25% of the original weight was lost, which corresponds to more than ¾ of the additive being dissolved and removed from the adhesive film.

It is expected that at “in-vivo” conditions the removed soluble phase in the adhesive film will create pathways for tissue growth connecting the bonded surfaces, leading to quick healing. It is also expected that at “in-vivo” conditions the rate of removal of the soluble phase will be quicker compared to “in-vitro” conditions.

### EXAMPLE 4

### Scanning Electron Microscopy

Scanning electron microscopy was used to observe the changes taking place at the surface of the adhesive films. The photographs clearly show the formation of voids as large as 100 microns within the films of the adhesives of the present invention as early as the sixth day following immersion in the buffer (photos 1 and 3). The film surface after 171 days clearly shows erosions and pathways with dimensions greater than 100 microns (photos 2 and 4). For comparison adhesive film based on unmodified NBCA (Photos 5 and 6) has featureless appearance without any erosions, pores or pathways.

### EXAMPLE 5

### In-vitro Strength Loss of Bonded Joints

It is essential that besides enhanced bioresorbability the adhesives of the present invention retain their adhesive strength with time and are capable of keeping the adhesively bonded tissues together.

Pieces of polyamide mesh were cut in 10 cm length and 2.54 cm width and were acetone soaked and washed to remove any finishing agents. The mesh pieces were left to dry at room temperature for 24 hours. Adhesive was placed on one piece, which was then overlapped at 1.27 cm with another piece. The adhesive was left to cure for 24 hours. The bonded mesh samples were placed in phosphate buffer solution of pH=7.2, conditioned and kept at 37° C. Samples were taken out at predetermined intervals, washed with deionized water and placed in an oven to dry for 24 h at 37° C. After annealing to room temperature the bonded assembly was tested by pulling to failure on an Instron machine with 1 mm/min crosshead speed. Each data is an average of 5 measurements and is presented in Table 4. The results demonstrate that the adhesives of the present invention maintain sufficient adhesive strength with time in “in-vitro” test media.

TABLE 4
In-vitro strength loss of adhesives

| | |
|---|---|
| Days in buffer | Tensile shear strength (N/mm²) |

| at 37° C. | Adh 2 | Adh 4 | Adh 6 | Adh 7 |
|---|---|---|---|---|
| 0 | 0.70 | 0.35 | 0.66 | 0.40 |
| 45 | 0.57 | 0.25 | 0.50 | 0.30 |
| 104 | 0.52 | 0.20 | 0.45 | 0.20 |
| 171 | 0.47 | 0.16 | 0.43 | 0.12 |

Other embodiments and uses of the invention will be apparent to those skilled in the art from consideration of the specification and practice of the invention disclosed herein. All references cited herein, including patents, are specifically and entirely incorporated by reference. It is intended that the specification and examples be considered exemplary only, with the true scope and spirit of the invention indicated by the following claims.

What is claimed as new and desired to be secured by Letters Patent of the United States is:

1. A bioresorbable adhesive composition comprising:
i. a cyanaocrylate monomer and,
ii. a body fluid soluble additive.

2. The composition of claim 1 wherein the body fluid soluble additive is selected from the group consisting of calcium L(+) lactate, magnesium L(+) lactate, gluconic acid delta lactone, ε-caprolactone, soluble starch, gelatin, innulin from chicory leaf, 2-hydroxycaproic acid and mixtures thereof.

3. The composition of claim 1 wherein the body fluid soluble additive is selected from the group consisting of mixtures of the calcium salts of L(+) lactic acid, gelatin, innulin, and mixtures of the above with c-caprolactone and 2-hydroxycaproic acid.

4. The composition of claim 1 wherein the body fluid soluble additive is selected from the group consisting of mixtures of the calcium salts of L(+) lactic acid with 6-caprolactone and mixtures of innulin with c-caprolactone.

5. The composition of claim 1 wherein the cyanoacrylate monomer is selected from the group consisting of alkyl 2-cyanoacryl ate, alkenyl 2-cyanoacrylate, alkoxyalkyl 2-cyanoacryl ate, carbalkoxyalkyl 2-cyanoacrylate and mixtures thereof.

6. The composition of claim 5 wherein the alkyl group of the cyanoacrylate monomer or monomers includes cycloalkyl groups.

7. The composition of claim 6 wherein the alkyl group of the cyanoacrylate monomer or monomers has from 1 to 16 carbon atoms inclusive.

8. The composition of claim 1 wherein the cyanoacrylate monomer is selected from the group consisting of methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate, 2-propoxyethyl 2-cyanoacrylate and mixtures thereof.

9. The composition of claim 1 further comprising a copolymer derived from one or more cyanoacrylate monomers and one or monomers selected from the group consisting of glycolide, lactide, ε-caprolactone, dioxanone and trimethylene carbonate.

10. The composition of claim 9 wherein the cyanoacrylate of the said copolymer is selected from the group consisting of alkyl 2-cyanoacrylate, alkenyl 2-cyanoacrylate, alkoxyalkyl 2-cyanoacryl ate, carbalkoxyalkyl 2-cyanoacrylate and mixtures thereof.

11. The composition of claim 10 wherein the alkyl group of the cyanoacrylate monomer or monomers of the said copolymer has from 1 to 16 carbon atoms inclusive.

12. The composition of claim 9 wherein the cyanoacrylate monomer of the said copolymer is selected from the group consisting of methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate, 2-propoxyethyl 2-cyanoacrylate and mixtures thereof.

13. The composition of claim 1 further comprising a copolymer derived from glycolide and one or monomers selected from the group consisting of lactide, r-caprolactone, dioxanone and trimethylene carbonate.

14. The composition of claim 1 wherein
i. the cyanoacrylate monomer is selected from the group consisting of methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate, 2-propoxyethyl 2-cyanoacrylate and mixtures thereof and
ii. wherein the body fluid soluble additive is selected from the group consisting of mixtures of the calcium salts of L(+) lactic acid with F-caprolactone and mixtures of innulin with ε-caprolactone.

15. The composition of claim 1 wherein the size of the additive particles is from about 0.5 μ to about 1000 μ.

16. The composition of claim 1 wherein the size of the additive particles is from about 10 μ to about 500 μ.

17. The composition of claim 1 wherein the size of the additive particles is from about 50 μ to about 300 μ.

18. The composition of claim 1 wherein the rate of bioresorption of the cured adhesive is characterized by a loss of at least about 6% of the mass of the adhesive within the first 7 days after application.

19. The composition of claim 1 wherein the rate of bioresorption of the cured adhesive is characterized by a loss of at least about 10% of the mass of the adhesive within the first 7 days after application.

20. The composition of claim 1 wherein the rate of bioresorption of the cured adhesive is characterized by a loss of at least about 20% of the mass of the adhesive within the first 7 days after application.

21. A method of making a bioresorbable adhesive comprising dispersing a body fluid soluble additive in a cyanoacrylate monomer.

22. The method of claim 21 wherein the body fluid soluble additive is selected from the group consisting of calcium L(+) lactate, magnesium L(+) lactate, gluconic acid delta lactone, ε-caprolactone, soluble starch, gelatin, innulin from chicory leaf, 2-hydroxycaproic acid and mixtures thereof.

23. The method of claim 21 wherein the body fluid soluble additive is selected from the group consisting of mixtures of the calcium salts of L(+) lactic acid, gelatin, innulin, and mixtures of the above with ε-caprolactone and 2-hydroxycaproic acid.

24. The method of claim 21 wherein the body fluid soluble additive is selected from the group consisting of mixtures of the calcium salts of L(+) lactic acid with F-caprolactone and mixtures of innulin with ε-caprolactone.

25. The method of claim 21 wherein the cyanoacrylate monomer is selected from the group consisting of alkyl 2-cyanoacrylate, alkenyl 2-cyanoacrylate, alkoxyalkyl 2-cyanoacrylate, carbalkoxyalkyl 2-cyanoacrylate and mixtures thereof.

26. The method of claim 25 wherein the alkyl group of the cyanoacrylate monomer or monomers includes cycloalkyl groups.

27. The method of claim 26 wherein the alkyl group of the cyanoacrylate monomer or monomers has from 1 to 16 carbon atoms inclusive.

28. The method of claim 21 wherein the cyanoacrylate monomer is selected from the group consisting of methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate, 2-propoxyethyl 2-cyanoacrylate and mixtures thereof.

29. The method of claim 21 further comprising dissolving a copolymer derived from one or more cyanoacrylate monomers and one or monomers selected from the group consisting of glycolide, lactide, F-caprolactone, dioxanone and trimethylene carbonate.

30. The method of claim 29 wherein the cyanoacrylate of the said copolymer is selected from the group consisting of alkyl 2-cyanoacrylate, alkenyl 2-cyanoacrylate, alkoxyalkyl 2-cyanoacrylate, carbalkoxyalkyl 2-cyanoacrylate and mixtures thereof.

31. The method of claim 30 wherein the alkyl group of the cyanoacrylate monomer or monomers of the said copolymer has from 1 to 16 carbon atoms inclusive.

32. The method of claim 29 wherein the cyanoacrylate monomer of the said copolymer is selected from the group consisting of methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate, 2-propoxyethyl 2-cyanoacrylate and mixtures thereof.

33. The method of claim 21 further comprising dissolving a copolymer derived from glycolide and one or monomers selected from the group consisting of lactide, ε-caprolactone, dioxanone and trimethylene carbonate.

34. The method of claim 21 wherein
i. the cyanoacrylate monomer is selected from the group consisting of methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate, 2-propoxyethyl 2-cyanoacrylate and mixtures thereof and
ii. wherein the body fluid soluble additive is selected from the group consisting of mixtures of the calcium salts of L(+) lactic acid with F-caprolactone and mixtures of innulin with ε-caprolactone.

35. The method of claim 21 wherein the size of the additive particles is from about 0.5 μ to about 1000 μ.

36. The method of claim 21 wherein the size of the additive particles is from about 10 μ to about 500 μ.

37. The method of claim 21 wherein the size of the additive particles is from about 50 ∞ to about 300 μ.

38. A method of treating living tissue comprising: applying to living tissue a bioresorbable adhesive composition comprising at least one cyanoacrylate monomer and a body fluid soluble additive.

39. The method of claim 38 wherein the body fluid soluble additive is selected from the group consisting of calcium L(+) lactate, magnesium L(+) lactate, gluconic acid delta lactone, ε-caprolactone, soluble starch, gelatin, innulin from chicory leaf, 2-hydroxycaproic acid and mixtures thereof.

40. The method of claim 38 wherein the body fluid soluble additive is selected from the group consisting of mixtures of the calcium salts of L(+) lactic acid, gelatin, innulin, and mixtures of the above with ε-caprolactone and 2-hydroxycaproic acid.

41. A method of claim 38 wherein the body fluid soluble additive is selected from the group consisting of mixtures of the calcium salts of L(+) lactic acid with c-caprolactone and mixtures of innulin with ε-caprolactone.

42. A method of claim 38 wherein the cyanoacrylate monomer is selected from the group consisting of alkyl 2-cyanoacrylate, alkenyl 2-cyanoacrylate, alkoxyalkyl 2-cyanoacrylate, carbalkoxyalkyl 2-cyanoacrylate and mixtures thereof.

43. A method of claim 42 wherein wherein the alkyl group of the cyanoacrylate monomer or monomers includes cycloalkyl groups.

44. A method of claim 43 wherein the alkyl group of the cyanoacrylate monomer or monomers has from 1 to 16 carbon atoms inclusive.

45. A method of claim 38 wherein the cyanoacrylate monomer is selected from the group consisting of methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate, 2-propoxyethyl 2-cyanoacrylate and mixtures thereof.

46. A method of claim 38 wherein the body fluid soluble additive is from about 1% by weight to about 50% by volume of the adhesive composition.

47. A method of claim 38 wherein the adhesive when cured is capable of rapid bioresorption leading to the formation of pores and pathways in the adhesive layer facilitating connective tissue growth.

48. A method of claim 38 wherein the rate of bioresorption of the cured adhesive is characterized by a loss of at least 6% of the mass of the adhesive within the first 7 days after application.

49. A method of claim 38 wherein the rate of bioresorption of the cured adhesive is characterized by a loss of at least 10% of the mass of the adhesive within the first 7 days after application.

50. A method of claim 38 wherein the rate of bioresorption of the cured adhesive is characterized by a loss of at least 20% of the mass of the adhesive within the first 7 days after application.

51. An adhesive composition comprising:
i. at least one cyanoacrylate monomer, and
ii. a body fluid soluble additive,
which when cured is capable of rapid bioresorption leading to the formation of pores and pathways in the adhesive layer facilitating connective tissue growth.

52. An adhesive composition as in claim 51 wherein the body fluid soluble additive is selected from the group consisting of calcium L(+) lactate, magnesium L(+) lactate, gluconic acid delta lactone, ε-caprolactone, soluble starch, gelatin, innulin from chicory leaf, 2-hydroxycaproic acid and mixtures thereof.

53. An adhesive composition as in claim 51 wherein the body fluid soluble additive is selected from the group consisting of mixtures of the calcium salts of L(+) lactic acid, gelatin, innulin, and mixtures of the above with c-caprolactone and 2-hydroxycaproic acid.

54. An adhesive composition as in claim 51 wherein the body fluid soluble additive is selected from the group consisting of mixtures of the calcium salts of L(+) lactic acid with F-caprolactone and mixtures of innulin with ε-caprolactone.

55. An adhesive composition as in claim 51 wherein the cyanoacrylate monomer is selected from the group consisting of alkyl 2-cyanoacrylate, alkenyl 2-cyanoacrylate, alkoxyalkyl 2-cyanoacrylate, carbalkoxyalkyl 2-cyanoacrylate and mixtures thereof.

56. A adhesive composition as in claim 55 wherein wherein the alkyl group of the cyanoacrylate monomer or monomers includes cycloalkyl groups.

57. A adhesive composition as in claim 56 wherein the alkyl group of the cyanoacrylate monomer or monomers has from 1 to 16 carbon atoms inclusive.

58. A adhesive composition as in claim 51 wherein the cyanoacrylate monomer is selected from the group consisting of methyl 2-cyanoacrylate, ethyl 2-cyanoacrylate, n-propyl 2-cyanoacrylate, iso-propyl 2-cyanoacrylate, n-butyl 2-cyanoacrylate, iso-butyl 2-cyanoacrylate, hexyl 2-cyanoacrylate, n-octyl 2-cyanoacrylate, 2-octyl 2-cyanoacrylate, 2-methoxyethyl 2-cyanoacrylate, 2-ethoxyethyl 2-cyanoacrylate, 2-propoxyethyl 2-cyanoacrylate and mixtures thereof.

59. A adhesive composition as in claim 51 wherein the body fluid soluble additive is from about 1% by weight to about 50% by volume of the adhesive composition.

60. An adhesive composition as in claim 51 wherein the rate of bioresorption of the cured adhesive is characterized by a loss of at least about 6% of the mass of the adhesive within the first 7 days after application.

61. An adhesive composition as in claim 51 wherein the rate of bioresorption of the cured adhesive is characterized by a loss of at least about 10% of the mass of the adhesive within the first 7 days after application.

62. An adhesive composition as in claim 51 wherein the rate of bioresorption of the cured adhesive is characterized by a loss of at least about 20% of the mass of the adhesive within the first 7 days after application.

63. An adhesive composition as in claim 51 wherein the pores created by the bioresorption of the cured adhesive are from about 0.5 μ to about 1000 μ in size.

64. An adhesive composition as in claim 51 wherein the pores created by the bioresorption of the cured adhesive are from about 10 μ to about 500 μ in size.

65. An adhesive composition as in claim 51 wherein the pores created by the bioresorption of the cured adhesive are from about 50 μ to about 300 μ in size.
